(19) United States
(12) Patent Application Publication
(10) Pub. No.: US 2006/0292550 A1
(43) Pub. Date: Dec. 28, 2006

(54) Method for determining fish composition using bioelectrical impedance analysis

(76) Inventor: Marlin Keith Cox, Sitka, AR (US)

Correspondence Address: Alex Nolte, 24610 Stoughton Ct., Katy, TX, 77494, US

(21) Appl. No.: 11/474,570

(22) Filed: Jun. 26, 2006

Related U.S. Application Data
(60) Provisional application No. 60/693,865, filed on Jun. 24, 2005.

(51) Int. Cl. C12Q 1/00

(52) U.S. Cl. 435/4; 205/777.5

(57) ABSTRACT

A method for determining the freshness of fish. The method includes applying electrodes to the fish, passing an electrical current between the electrodes, determining an impedance for tissue of the fish between the electrodes, and correlating the determined impedance with a freshness value to determine the freshness of the fish.

### CROSS REFERENCE TO RELATED APPLICATIONS

This application claims priority under 35 USC §120 to U.S. Provisional Patent Application Ser. No. 60/693,865, which was filed on Jun. 24, 2006. The contents of this provisional application are incorporated by reference into the present application in its entirety.

### BACKGROUND OF THE INVENTION

1. Field of the Invention

Embodiments of the invention generally relate to composition analysis techniques for determining the quality or freshness of fish.

2. Description of the Related Art

Currently, there are several methods for determining biological composition of fish, however, these conventional methods are generally impractical for use outside of a laboratory setting as a result of the efficiency and costs involved in the composition analysis. For example, direct measurement of individual compartments using chemical analysis requires sacrificing the specimen, followed by lengthy laboratory procedures to determine the freshness or quality of the fish. Total body water, another conventional method for gauging the health of a fish, is a good predictor of other body composition estimations, but in vivo methods to estimate TBW involve using radioisotope tracers such as tritium, deuterium and oxygen-18. These markers are difficult to analyze outside a highly specialized laboratory and are cost prohibitive, which once again precludes mass use for determining the quality or freshness of fish. Fat free mass can be estimated non-sacrificially by counting intracellular potassium-40 with thallium activated sodium iodide crystal detectors, but again, the high cost, instrumentation required, and technicality diminishes its practicality for use in any sort of day to day operation.

Electrical conductivity methods for measuring biological composition are non-sacrificial and include total body electrical conductivity (TOBEC) and bioelectrical impedance analysis (BIA). These methods generally rely on impedance differences between fat and fat-free tissues. The TOBEC method was found to accurately estimate composition for healthy individuals, but measurement errors increased substantially when the measurement subjects were undergoing weight or compositional changes, or when subjects with dissimilar body sizes were compared, as conductivity is known to change with geometry and size. Furthermore, the TOBEC unit is large and non-portable, making field use impractical.

Therefore, a non-invasive and not fatal method of accurately determining energy density and body composition, which are directly related to fish quality and freshness, in fish is desired. Such a method would benefit bioenergetic modeling and the study of fish condition and growth and would provide a previously unavailable barometer for fish as a consumable product.

### SUMMARY OF THE INVENTION

Embodiments of the present invention generally provide a non-invasive and non fatal method of accurately determining energy density and body composition in fish. The method of the invention provides an easy to use, reliable, and cost effective method for measuring the quality or freshness of fish in a commercial environment.

Embodiments of the invention may further provide a method for determining the freshness of fish. The method includes applying at least four electrodes to the fish, passing an electrical current between at least two of the at least four electrodes, determining an impedance for tissue of the fish between electrodes, and correlating the determined impedance with a freshness value to determine the freshness of the fish.

Embodiments of the invention may further provide a method for determining the quality of fish. The method includes passing a current between at least four electrodes positioned in communication with the fish, measuring an impedance of the fish from the passing of the current, and determining a freshness of the fish by correlating the measured impedance to a model of the particular species of fish being tested.

Embodiments of the invention may further provide a method for determining freshness of fish. The method generally includes passing an electrical current between at least four electrodes positioned in communication with the fish, wherein the current is a constant alternating current of between about 500 μA and about 900 μA at about 50 Hz, and correlating an impedance measured from the passing of the electrical current to a model of the particular species of fish being tested, wherein the model is an empirically generated independent predictive model for the species, and wherein the independent predictive model correlates the measured impedance to total body water, dry weight, fat-free mass, total body protein, total body ash, or total body fat of the fish species to determine the freshness of the fish.

### BRIEF DESCRIPTION OF THE DRAWINGS

So that the manner in which the above recited features of the present invention can be understood in detail, a more particular description of the invention, briefly summarized above, may be had by reference to embodiments, some of which are illustrated in the appended drawings. It is to be noted, however, that the appended drawings illustrate only typical embodiments of this invention and are therefore not to be considered limiting of its scope, for the invention may admit to other equally effective embodiments.

FIG. 1 illustrates a diagram of the electrode placement on a subject fish for analysis using an exemplary method of the invention;

FIG. 2 illustrates a simplified diagram of an exemplary BIA apparatus that may be used to implement one or more of the exemplary embodiments of the invention; and

FIG. 3 illustrates a flowchart of an exemplary methodology of the invention.

### DETAILED DESCRIPTION

The present invention generally provides a method for rapidly determining body composition of a fish using bioelectrical impedance analysis (BIA). The method generally includes using the electrical conductivity relationships of the tissue of the fish to determine parameters such as total body water (TBW), dry weight (DW), fat-free mass (FFM), total body protein (TBP), total body ash (TBA), total body fat (TBF), and any other tissue parameter that may be extrapolated or determined from a BIA-type analysis. These parameters may then be used to determine or extrapolate the freshness or quality of the fish.

Generally speaking, growth is considered the primary expression of the well being of a fish. Further, growth has also been directly linked to the reproductive success of a fish. Growth is known to reflect changes in the body composition or mass of growth components, e.g. fat and protein, relative to inert or compensating components, e.g., ash and water. To measure and determine the above noted parameters, embodiments of the invention provide independent predictive models configured to determine TBW, DW, FFM, TBP, TBA, and TBF using derivatives of resistance and reactance equations and input from a BIA method measurement. However, prior to discussing the exemplary embodiments and methodology of the intention, it is best served to first discuss the principles of BIA.

In BIA, proximate composition estimations are calculated by measuring the impedance, i.e., the real (resistance) and imaginary (reactance) components, of a current passed through a fish and regressing the dish with actual proximate body composition numbers for that fish. Resistance of a substance is known to be proportional to the voltage of an applied current as it passes through a substance (Ohm's Law), or
R=V/Ct, (1)

where R is resistance (ohms), V is applied voltage (volts), and Ct is current (amps). The reactance (X_{c}) is the opposition to alternating current by a capacitor (in the case of fish measurements, the cell membranes of the fish tissues form the capacitors, as will be further discussed below), and can be mathematically expressed by the following equation:
X_{c}=1/(2nfC) (2)

where X_{c} is reactance in Ohms, f is frequency in Hertz, and C is capacitance in Farads. Impedance (R and/or X_{c}) is related to the cross sectional area and conductor length of the fish and the signal frequency of the current. Impedance can be expressed by the equation:
I=ρL/A, (3)

where I is resistance or reactance, ρ is resistivity constant, L is measured length, and A is area (mm²). If the signal frequency and the configuration are held constant, the impedance measurements can be related to its volume. This is demonstrated by the following: if
R=ρL/A, (4)

is multiplied by UL, then
R=ρL²/V_{ε}. (5)

Using standard substitution methods,
V_{ε}=ρL²/R, (6)

where V_{ε} is volume (mm³) and p is determined statistically by regressions of V_{ε} on impedance.

Cell membranes of fish, as briefly mentioned above, consist of a non-conductive lipid bi-layer sandwiched between two conductive protein layers. These layers, at low voltages and high frequencies, such as those typically used in embodiments of the invention (for example, about 800 μA, AC, and 50 kHz, or between about 500 μA and about 900 μA), pass current mainly through the extra cellular fluids, while at higher frequencies the cell walls become capacitive. Therefore, at high frequency, reactance and resistance numbers to be sensitive to changes in volume of extra cellular and cellular material, which forms the baseline for determining the freshness or quality of the cells, as the change in volume of extra cellular and cellular material has been shown to be related to cell health or quality.

The measurement method of the invention generally includes placing electrodes on the subject fish, passing a current between the electrodes, and taking measurements related to the current, or more particularly the resistance to the current, traveling between the electrodes. Thus, the electrode placement on the subject fish is the first and primary step of the exemplary methods of the invention. Electrode placement can fall under measures for body composition and condition. Body composition includes measurements of fat, fat-free, dry, protein, water and ash masses. Once a model is made for each of these parameters for each type or species of fish being measured, further measurements of fish will provide the user with very accurate indications of these parameters in a mass unit (e.g., grams). Body composition measurements are sensitive to needle size, depth, and position; and therefore, once a model is created using a certain needle size, depth and position, all subsequent measurements should also be made using parameters similar to the model creation parameters to allow for accurate and repeatable measurements.

As an example, the needles typically used for BIA measurements in embodiments of the invention are 28 gauge stainless steel electrodes and are generally inserted about 1 cm deep. In some embodiments of the invention, the needle is inserted between about 1 mm and about 6 mm deep for the BIA measurement process. A variety of needle sizes could be used, but measurements will vary with needle gauge, as a result of the electrode interface with the fish tissue, and therefore, once a model is developed for a species of fish with a particular size needle, subsequent measures on that species of fish should be with the same needle gauge for the model data to apply. Likewise, if more or less of the needle is inserted than with the model measurements, there will be a change in the electrode interface, and the results of the BIA measurements will not be the same as the model measurements. Thus, not only is using the same needle size important, but also using the same needle insertion depth is important, as using a different needle depth from that which was used in the model measurements will generally impact the accuracy of the BIA measurements.

Further, the position of the needle/electrode on the subject fish is sensitive to the area of the fish being measured. Similarly, if the model is developed on a certain section of the fish, subsequent measurements should be from the same body location as the model. Also, each species of fish may require an independent model to be made, so electrode placements should be consistent within fish of a particular species. This is due to BIA measurements representing the section of fish between the detecting electrodes. If needles are moved 1 mm, 1 cm, 3 cm, etc. from the desired position, BIA will be measuring a different segment of the fish and the model measurements will not be accurate. If, for example, a user wanted to measure belly flap fat in a first type of fish, the appropriate location of electrodes would be in the belly section and not the dorsal or head region, whereas if the user wanted a dorsal systemic measure of fat content, the user would place electrodes on the dorsal section of the fish where systemic fat is located and not the ventral section.

As a note, the inventors have successfully used both dorsal and ventral measurements in measuring body composition of fish. Measurements were used to measure these different aspects of fish and specifically, 1) dorsal BIA measurements were used to measure body composition in the dorsal aspects of fish and 2) ventral measurements were used to measure body composition in the ventral aspects of fish as well determining egg and/or sperm content.

In fish measured dorsally, electrode placements are generally as follows. The anterior most electrode (signal) is placed at about the midpoint between the apex of the operculum plate and the nape of the dorsal side of the fish. The posterior most electrode (signal) is placed midpoint between the lateral line and the adipose fin. The detecting electrodes of each set are placed about one cm inside of each signal electrode. The one cm generally represents a measurement standard that was adhered to for generating the model, however, the invention is not intended to be limited to any particular spacing outside of that which supports accurate and repeatable measurements. In a typical embodiment of the invention, all four electrodes are generally placed in a generally linear manner along the side of the subject fish. These particular electrode placement areas were selected for the dorsal systemic musculature of the fish, and the lack of vital organs in the areas where insertion of electrodes would harm the fish.

In fish measured ventrally, electrode placements are generally as follows. The anterior most electrode (signal) is placed on the area just behind the area where the gills come together. The posterior (signal) electrode is placed to one side of the anus vent. The detecting electrodes are again placed about one cm inside of each signal electrode, and again, the electrodes are generally in line with one another in a typical embodiment of the invention.

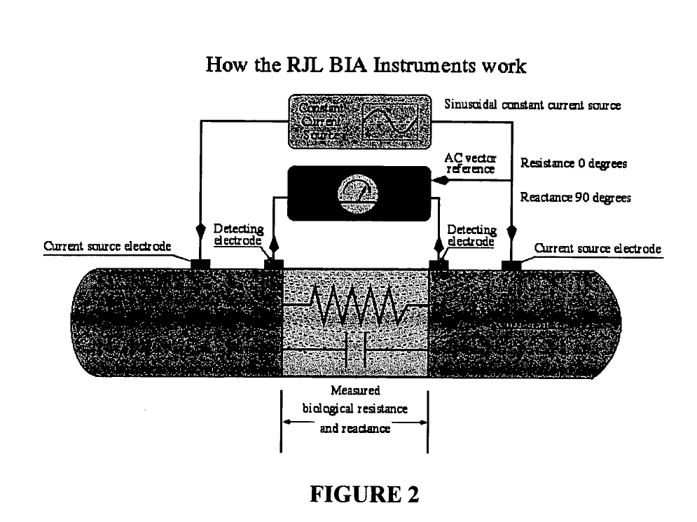

FIG. 2 illustrates a diagram of an exemplary BIA apparatus that may be used to implement embodiments of the invention.

With the electrodes positioned, condition measurements can be made from taking the phase angle (α) where α=arctan*(reactance/resistance). Phase angle is a linear method of measuring the relationship between resistance and reactance in series or parallel circuits. The phase angle can range from 0 to 90 degrees; 0 degrees if the circuit is only resistive (as in a system with no cell membranes) and 90 degrees if the circuit is only capacitive (all membranes with no fluid). A phase angle of 45 degrees would reflect a circuit (or body) with an equal amount of capacitive reactance and resistance. Lower phase angles are generally consistent with low reactance and either cell death or a breakdown in the selective permeability of the cell membrane. Higher phase angles are generally consistent with high reactance and large quantities of intact cell membranes and body cell mass. Both resistance and reactance measurements are in Ohms, and therefore, when one is divided by the other, you get a unitless value. The arctan portion forces the numbers between 0-90°, so the result is a unitless gauge-type measure. This allows needle size, depth and position to be variable because the measurement units cancel each other out.

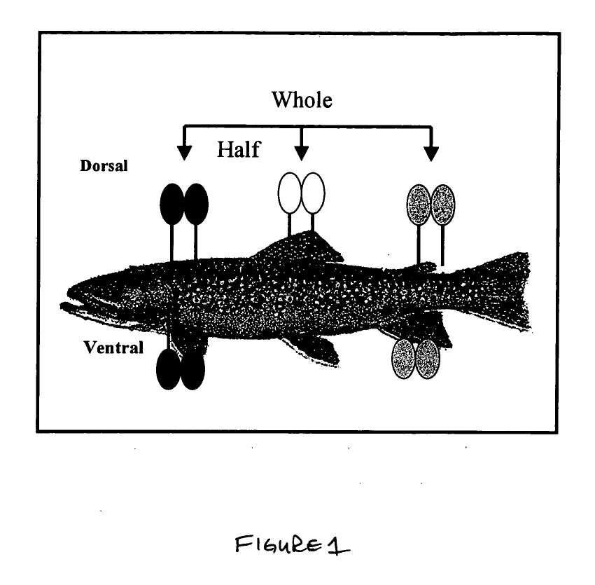

Regressing back to the methodology of the measurement process of an exemplary method of the invention, with the electrodes positioned, the method continues to the next step where the measurement signal is passed through the subject fish. More particularly, whole body electrical impedance (real and imaginary/resistance and reactance) is measured by passing a small constant alternating current through the body of the subject fish between the electrodes and measuring the voltage drop produced as a product of resistance and current. Since current is generally constant, the voltage is known to be directly proportional to the resistance. A shift in the phase angle between the current and voltage defines reactance or a complex impedance measurement including the dielectric non-conducting space attributed to cell membrane capacitance. Electrode placement, measurement frequency, and skin impedance are the primary procedural specifications that must accompany impedance data. Skin impedance ranges from approximately 300 to one million ohm/cm, and therefore, in order to accurately assess body volume electrically, skin impedance should be bypassed using either the two electrode or four electrode techniques illustrated in FIG. 1.

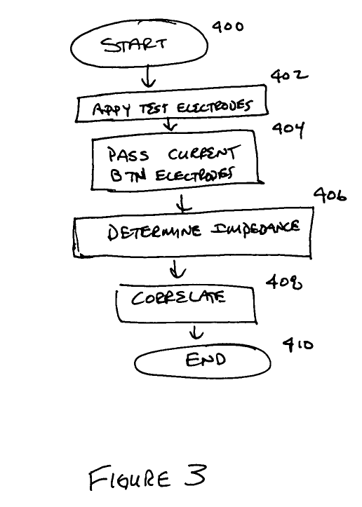

FIG. 3 illustrates a flowchart of an exemplary method of the invention. The method begins at step 400 and continues to step 402, wherein the test electrodes are applied to the subject fish. Once the electrodes are applied, the method continues to step 404, where an electrical current is passed between the electrodes. At step 406 of the exemplary method, the impedance between the electrodes is determined. Thereafter, the method continues to step 408, wherein the determined impedance correlated with a freshness value to determine the freshness of the fish. The correlation step may include generating a model for the type of fish being tested and comparing the determined impedance parameter, or a parameter calculated from the impedance parameter, to the model to determine the freshness of the fish. The model may be generated through empirical measurements of the same type of fish to determine a relationship between a measured impedance of the fish and the quality of the fish tissue. The exemplary method of the invention then ends at step 410.

With regard to the electrode placement, Applicant notes that the two electrode technique used by conventional measurement methods has several limitations. Results from conventional electrode placement techniques are often irreproducible due to excessive interference by electrochemical reactions at the subcutaneous needle electrode surface causing additional electrode polarization anomalies. Additionally, the small diameter of the electrode needles results in a much greater current density near the electrodes than in the rest of the body.

Therefore, the integrity of tissue near the electrodes and electrode size can effect the impedance measurement between electrodes, and confuse desired data. The four electrode techniques used in embodiments of the invention generally avoids these difficulties. Four surface electrodes are used situated either ipsilaterally or contralaterally on the dorsal surfaces of the right hand and foot at the distal metacarpals and metatarsals, respectively, and the distal prominences of the radius and ulnar and between the medial and lateral malleoli at the ankle. The BIA system. which may be a system such as those manufactured by RJL Inc., may deliver about 800 uA at 50 KHz between the outer two electrodes. The voltage drop between the inner two electrodes is measured with a high input impedance amplifier. The impedance of the skin and the electrode polarization impedance does not effect the measurement of total body impedance with the four surface electrode technique, since negligible current is drawn through the skin by the passively coupled input circuits. The four surface electrode technique utilizing a constant deep homogeneous electrical field also minimizes problems with field distribution and electrode irregularities. The constant current source is generally regulated to ±1% accuracy from 0 to 8,000 ohms, and the detecting electrodes have input characteristics that do not require complex electrodes or conductive bands.

Further, body composition formulas are utilized to determine the bioeugenic characteristics of the fish. More particularly, for each individual species or type of fish, a model is made for each characteristic measured, e.g., fat, fat-free, protein, water, dry and ash masses. The actual measurements (from the lab analysis) are regressed with each of the 6 equations, and the the best fit is then used as the model. Because of the inherent, yet minimal, uncertainty of how electrical currents move through biological tissue, a best fit model approach is used within the boundaries of the following six equations.

| Number | ID | Equation | Description |
|---|---|---|---|
| 1 | E1 | len_{d}²/R | Resistance series |
| 2 | E2 | len_{d}²/Rₚ | Resistance parallel |
| 3 | E3 | len_{d}²/X_{c} | Reactance series |
| 4 | E4 | len_{d}²/X_{cp} | Reactance parallel |
| 5 | E5 | len_{d}²/C_{pf} | Capacitance in |
| | | | farads |
| 6 | E6 | len_{d}²/Z | Phase angle |
| | | | resistance and |
| | | | reactance in parallel |

In the above listed 6 equations, len_{d} generally represents the detector length, R generally represents the resistance, and X_{c} generally represents the reactance. Therefore, using these parameters, the following equations are presented:
Rₚ=R+(X_{c}²/R);
X_{cp}=X_{c}+(R²/X_{c});
C_{pf}=(1*10⁻¹²)/(2*3.14*50000*X_{cp}); and
Z=sqrt(R²+X_{c}²).
As generally noted above, phase angle is the parameter that is generally used in embodiments of the invention to measure and determine the condition of the tissue of the fish, as the quantity and efficiency of cells in organic tissues, and in particular in fish tissue, has been shown to be directly proportional to the phase angle in a BIA-type analysis. More particularly, the outer boundary of the fish cell is a plasma membrane of phospholipid molecules that are a dielectric, and as such, this forms an electrical capacitor when a radio frequency signal is introduced to the cell environment. Capacitance is fundamental to any organic tissue phase angle measurement, i.e., the higher the capacitance the greater the phase angle. For example, an elite athlete would generally have a higher phase angle measurement of tissue than a measurement obtained from the tissue of a sedentary person. It has been well documented that phase angle declines with disease, age, and reduced activity level, and as such, Applicant submits that the measure of the phase angle of fish tissue may be used in the method of the present invention to determine the condition, age, disease level, etc. of fish in a commercial environment, although this measurement has not been undertaken in conventional methods. The application of the method of the invention may be used for commercial hatcheries, fish farms, and even at the market level to determine the age and quality of fish for sale for consumption.

Returning to the methodology of the invention, before measurement of a fish, the fish is generally anesthetized, or in the case where the fish being measured is a food product, anesthetization is not required. Once the fish is anesthetized, the fish may be measured with the BIA. Electrical impedance (resistance and reactance) is measured with, for example, a tetrapolar bioelectrical impedance analyzer, such as the analyzer commercially available from RJL Systems of Detroit, Mich. The analyzer has two sets of needle electrodes, which may be stainless steel, 28 gauge, and 12 mm in length, with each set consisting of one signal and one detecting electrode placed about 1.0 cm apart. One set of electrodes may be placed in the anteriad dorsad region of the fish, and the second set of electrodes may be placed in the caudal peduncle region of the fish, as illustrated in FIG. 1. Each set of electrodes is generally placed in a consistent position for each species, type, or classification of fish, as determined by the particular model for the species, type, or classification of fish tested.

For the fish tested in the exemplary embodiment of the invention, the causal electrodes are generally placed midway between the lateral line and dorsal midpoint, and the anterior set of electrodes positioned at the anterior apex of the operculum and the posterior set positioned even with the anterior edge of the adipose fin, as generally illustrated in FIG. 1. The electrode needles were positioned and configured to penetrate about 2 mm into the fish at each electrode location. The distance between the two detecting electrodes was measured for each fish, and a current was introduced through the signal electrodes and the proximal detecting electrodes measured the voltage drop in the signal received at the electrodes. These two electrical values, resistance and reactance, were then used to calculate values from common electrical property equations that included resistance in series and in parallel, reactance in series and in parallel, combined resistance and reactance in series and in parallel, and capacitance. These values were then used as independent variables in the regression models to determine the appropriate parameter of the fish, i.e., total body water, dry weight, fat-free mass, total body protein, total body ash, total body fat, and any other fish tissue related parameter that may be extrapolated or determined from a BIA-type analysis that may be useful in determining the quality of the fish tissue.

Independent models for each parameter were built from the “model” group. Dependent variables in the regression models were compared to the body composition values measured in empirical model building measurements, and independent variables were values from the electrical property equations. Relationships between the two variables (equations and actual parameter values) are explained with correlation analysis for strength of linear relationships and predictability, residual plots and F values (to test significance of slopes in linear regressions), and confidence limits on the regression coefficients (to test for 1:1 relationships) in the Cox Hartman paper mentioned below. Best-fit linear models are then used to predict parameter estimates and predicted values for each parameter are expressed by the following equation:
Pθ=(PP/PW)*weight,

where Pθ is the new predicted parameter value (g), PP is the initial predicted parameter (g), PW is the predicted total weight (g) and weight is the measured actual wet weight of the fish.

Further, measuring body composition using the BIA method of the invention allows individual fish to repeatedly be measured to follow compositional change as well as measuring fat (omega three), protein percentage, and other parameters that may be measured or modeled by BIA. A more detailed description of the analysis techniques and best fit linear models may be found in the publication entitled “Non-lethal estimation of proximate composition in fish,” by Marlin Cox and Kyle Hartman, which was published in the Canadian Journal of Fish and Aquatic Science in volume 62, page 269 on Mar. 12, 2005, the content of which is hereby incorporated by reference into the present application in its entirety, to the extent not inconsistent with the present invention.

Returning to the discussion of the methodology of the invention, strong linear relationships were found between independent variables calculated from BIA values, and observed body composition values. Independent and dependent variables with associated R values are as follows: resistance in series with TBW (R=0.9872), reactance (in parallel) with DW (R=0.9862), combined resistance and reactance in series with FFM (R=0.9873), resistance in series (R=0.9863), resistance in parallel with TBA (R=0.9864), and capacitance with TBF (R=0.9779). Additionally, tests of the linear regression models using a validation group indicated that the models were accurate indicators of all body composition values. Predicted and actual values for all body composition parameters were highly correlated (p<0.0001) with R² scores ranging from 0.8507 to 0.9986 in TBF and TBW, respectively. F tests (p<0.0001) and residuals revealed a linear relationship between impedance values and the predicted values. Correlations between predicted and observed values in all proximate composition categories indicated a strong linear relationship with values not differing from 1:1.

Linear regression models developed for fish, specifically, brook trout, have also been determined to be accurate predictors of DW, TBW and weight across all species of fish tested. Predicted values of DW, TBW and weight have also been strongly correlated with actual values. Dry weight predicted and actual values were highly correlated with R² ranging from 0.9537 in logperch to 0.9975 in smallmouth bass. Predicted and actual values of total body water were highly correlated with R²>0.9900. Predicted and actual total weight values were highly correlated with R²>0.9950 for other fish specimens. In repeated measurements testing the methodology of the invention, fish showed little response to being measured with BIA. Measured parameters of swimming, feeding, bleeding and color were not significantly different between two groups (applied BIA and a test group). Bruising was significant (logistic regression p<0.0001) in response to BIA, but it was observed to be slight and only lasted for two days.

The bioelectrical impedance analysis models generated for the fish tested in developing the method of the invention provided a means of estimating body composition in brook trout and a group of warm water fish species with a high degree of predictability (R²>0.8507). Additional experiments showed that the methods of BIA are non-lethal and appear to produce little measurable effect upon fish health or behavior. Using the apparatus of the invention, BIA estimations are derived from three measurements: resistance (measure of extracellular resistance), reactance (measurement of “celled” mass) and distance between detecting electrodes. These three measurements are used to predict TBW, DW, FFM, TBP, TBA and TBF, and are can be obtained on live, anaesthetized organisms in about the time it takes to measure live weight, i.e., in less than about 20 seconds. Similar measurements may be made on processed fish (no longer alive) in the same or less time.

The strong predictability and accuracy found in fish data is a result of the body geometry of fish being more simplified than the higher vertebrates used in conventional test methods. More specifically, fish (trout) have a fusiform geometric shape that approximates a cylinder with a majority of the mass located in the thorax. The thorax accommodates all major body composition components, and likewise, it is the main region for hypertrophic or hypotrophic growth. Since volume is proportional to impedance and length between detecting electrodes, a single impedance measurement represents the whole body and likewise, compositional changes that occur within it. If the majority of mass is not located within a single volume as with many higher vertebrates, it must be distributed into limbs or appendages. Since each limb or appendage has its own volume and tissue heterogeneity a single measurement of impedance cannot represent the whole organism (e.g. two different sized volumes with identical composition would have different impedance measurements). Likewise, measuring impedances for each separate volume and combining them would result in complex methodology and conversely a complex model with error propagations occurring with each volume. For a single measurement of impedance to be representative of the whole body, a simple body geometry with the majority of the mass located in one volume is essential.

A validation of a fish species BIA model with an independent BIA dataset showed the models accurately predicted actual values (R²>0.9277) except for lower predictability of fat mass (R²=0.8507). The reasoning for the weaker correlations for fat mass may be explained by electrical resistivity properties. The nature of current division described by Ohm's law and Kirchhoff's rules dictates that current will pass through an entire circuit, but the path with the least resistance will carry more current. Fat deposits are 1) concentrated in the ventral gut region of the fish and 2) have a higher resistance than other visceral or somatic tissues. Since electrode placement was in the dorsal region, and resistance is higher in fat, it is possible that the current does not represent the fat that is located in the lower ventral region. All other parameters such as TBW and TBP are more systemic, have a lower electrical resistance and therefore are better represented by impedance values in this study. This leads to the possibility of localized or tissue-specific BIA modeling via electrode placement.

Furthermore, strong linear relationships in a warm water species portion of a validation study indicated that the fish species used in the validation study model may predict compositional parameters for other species. This is generally due to the similar geometric shapes found among the species of fish used in the validation study. The warm water species validation group had of a cylindrical shape with the majority of the mass (body composition components) located within one volume, and predictions with the fish species for the BIA model were strongly correlated with actual parameter estimations. Some of the variation between predictions with the test species model and observed values for the warm water species, especially in the dry weight parameter may have been due to regional tissue deposits. It is believed that vertebrates such as fish, amphibians, reptiles and some mammals that fit the criteria mentioned above would be suitable for BIA modeling.

The ability to precisely and non-lethally estimate proximate composition through BIA will permit increased precision in energy flow and proximate composition studies on spatial and temporal scales that were previously impractical. At the individual level, BIA will permit repeated measures on the same individual during the course of investigation, yielding better tracking of energetics components and improved precision in bioenergetic models. At the population level, BIA will permit assessment of condition of cohorts over time and permit detailed comparisons across cohorts and temporal and spatial scales, such as evaluating body conditional properties of highly migratory species at different points during migration. At the community level, BIA will permit the evaluation of growth and energy flow dynamics across species. This capability may allow elucidation of community dynamics that were previously unknown, or permit correlation of body composition with outbreaks of disease. This approach also has potential for the non-lethal study of threatened or endangered species by using models developed for closely related species. Finally, this could be applied to other taxa, particularly amphibians and reptiles, with similar results and uses as described here.

TABLE I

| | | | | | | |
|---|---|---|---|---|---|---|
| TBW | 30 | 1.32045 | 3.46187 | L²/Rₘ | <0.0001 | 0.9746 |
| | | (1.94826) | (0.10560) | | | |
| DW | 30 | 0.29558 | 4.31575 | L²/X_{cp} | <0.0001 | 0.9726 |
| | | (0.77843) | (0.13679) | | | |
| FFM | 30 | −0.51881 | 0.99968 | L²/Rₘ | <0.0001 | 0.9747 |
| | | (0.56108) | (0.03041) | | | |
| TBP | 30 | −0.67352 | 0.89630 | L²/Rₘ | <0.0001 | 0.9727 |
| | | (0.52328) | (0.02836) | | | |
| TBA | 30 | 0.11516 | 0.11749 | L²/Rₚ | <0.0001 | 0.9730 |
| | | (0.06089) | (0.00370) | | | |
| TBF | 30 | −1.79087 | 1.80382⁻²³ | L²/X_{cp} | <0.0001 | 0.9563 |
| | | (0.32015) | (7.28985⁻²⁵) | | | |

Table 1 illustrates relationships between proximate body composition components and impedance equations for the test fish species (brook trout Salvelinus fontinalis) in the model group. Analysis results of the linear relationships between the measured bioelectrical impedance equations and actual numbers of proximate body composition components: total body water (TBW), dry weight (DW), fat-free mass (FFM), total body protein (TBP), total body ash (TBA), and fat mass (TBF). In the table, x represents the specific impedance equation providing the best-fit, where L=length, R=resistance, Z=impedance, X is reactance, subscript p or m represent parallel or series circuitry, and a and b represent the intercept and slope of the regression, respectively.

Table 2 illustrates a correlation of coefficient scores (R2) of predicted and actual parameter values including total body water (TBW), dry weight (DW), and total weight for various warm water species (drum, gizzard shad, longear sunfish, logperch, Moxostoma. spp., rockbass, sauger, and smallmouth bass) using the test species BIA model.

TABLE 2

| | | R² | | |
|---|---|---|---|---|
| Species | N | DW | TBW | Weight |
| Freshwater drum | 11 | 0.9947 | 0.9995 | 1.0000 |
| Gizzard shad | 15 | 0.9854 | 0.9982 | 1.0000 |
| Longear sunfish | 10 | 0.9960 | 0.9973 | 0.9993 |
| Logperch | 6 | 0.9537 | 0.9906 | 0.9952 |
| Moxostoma spp. | 11 | 0.9932 | 0.9989 | 1.0000 |
| Rockbass | 4 | 0.9973 | 0.9988 | 0.9999 |
| Sauger | 11 | 0.9956 | 0.9994 | 1.0000 |
| Smallmouth bass | 11 | 0.9975 | 0.9996 | 1.0000 |

Applicant submits that a valuable commercial use of the methodology of the invention is in the commercial fish industry, i.e., in measuring the quality of fish that is or will be sold to the consumer. For example, with the BIA method of the invention, (dead) fish can be measured to determine how long the fish has been dead or on ice, e.g., to determine the “freshness,” and the corresponding value of the fish. This is determined through the method of the invention, as the time fish spends on ice (the time since the fish was living) is proportional to the fish cell degeneration, which is measurable by the method of the invention. Therefore, the method of the invention provides a fish freshness measurement that may be used by fish markets, restaurants, and possibly even consumers to determine the freshness of the fish the respective parties are purchasing.

While the foregoing is directed to embodiments of the present invention, other and further embodiments of the invention may be devised without departing from the basic scope thereof, and the scope thereof is determined by the claims that follow.

1. A method for determining the freshness of fish, comprising:
applying at least four electrodes to the fish;
passing an electrical current between at least two of the at least four electrodes;
determining an impedance for tissue of the fish between electrodes; and
correlating the determined impedance with a freshness value to determine the freshness of the fish.

2. The method of claim 1, wherein applying at least four electrodes comprises applying two signal electrodes and two detecting electrodes, the at least four electrodes being applied in a substantially linear arrangement.

3. The method of claim 1, wherein passing the electrical current comprises passing a constant alternating current through the fish between the at least 4 electrodes.

4. The method of claim 1, wherein determining the impedance comprises measuring a voltage drop produced during the passing of the electrical current.

5. The method of claim 4, wherein determining the impedance comprises calculating the impedance as a product of resistance and current.

6. The method of claim 1, wherein correlating the determined impedance with a freshness value comprises comparing the determined impedance with a model for the particular fish being tested to determine the freshness of the fish.

7. The method of claim 1, further comprising determining a model for the fish being tested through empirical measurements that correlate a measured impedance value with a particular grade of fish tissue.

8. The method of claim 1, wherein the correlating comprises determining a phase angle from the impedance value, wherein lower phase angles are consistent with low reactance and poor cell freshness and higher phase angles are consistent with high reactance and large quantities of intact cell membranes and body cell mass.

9. The method of claim 1, wherein applying at least four electrodes comprises:
applying an anterior signal electrode positioned at about a midpoint between an apex of an operculum plate and a nape of a dorsal side of the fish;
applying a posterior signal electrode positioned midpoint between a lateral line and an adipose fin; and
applying two detecting electrodes positioned about one cm inside of each of the anterior and posterior signal electrodes.

10. A method for determining the quality of fish, comprising:
passing an electrical current between at least four electrodes positioned in communication with the fish;
measuring an impedance of the fish from the passing of the current; and
determining a freshness of the fish by correlating the measured impedance to a model of the particular species of fish being tested.

11. The method of claim 10, further comprising positioning at least four electrodes on the fish to facilitate the passing step, the at least four electrodes comprising an anterior signal electrode positioned at about a midpoint between an apex of an operculum plate and a nape of a dorsal side of the fish, a posterior signal electrode positioned midpoint between a lateral line and an adipose fin, and two detecting electrodes positioned about one cm inside of each of the anterior and posterior signal electrodes.

12. The method of claim 11, wherein passing the electrical current comprises passing a constant alternating current between the anterior signal electrode and posterior signal electrode and the two detecting electrodes.

13. The method of claim 12, wherein the constant alternating current comprises between about 500 μA and about 900 μA at about 50 Hz.

14. The method of claim 13, wherein measuring the impedance comprises

15. The method of claim 14, further comprising determining a phase angle from a resistance and a reactance determined from the impedance, wherein a lower determined phase angle is indicates cell death or a breakdown in the selective permeability of the cell membrane and a higher determined phase angle indicates large quantities of intact cell membranes and body cell mass.

16. The method of claim 15, wherein correlating the measured impedance to a model of the particular species of fish being tested comprises empirically generating an independent predictive model for a species of fish being tested, wherein the independent predictive model correlates the determined impedance to total body water, dry weight, fat-free mass, total body protein, total body ash, or total body fat of the fish species.

17. The method of claim 16, further comprising:
determining the distance between the respective electrodes;
measuring the voltage drop between the respective electrodes;
determining a resistance and reactance of tissue between the respective electrodes;
calculating values from common electrical property equations, the values including resistance in series and in parallel, reactance in series and in parallel, combined resistance and reactance in series and in parallel, and capacitance; and
using the values as independent variables in regression models to determine a freshness parameter of the fish.

18. The method of claim 17, wherein the freshness parameter comprises at least one of total body water, dry weight, fat-free mass, total body protein, total body ash, and total body fat

19. A method for determining freshness of fish, comprising:
passing an electrical current between at least four electrodes positioned in communication with the fish, wherein the current is a constant alternating current of between about 500 μA and about 900 μA at about 50 Hz;
correlating an impedance measured from the passing of the electrical current to a model of the particular species of fish being tested, wherein the model is an empirically generated independent predictive model for the species, and wherein the independent predictive model correlates the measured impedance to total body water, dry weight, fat-free mass, total body protein, total body ash, or total body fat of the fish species to determine the freshness of the fish.
